(19) United States
(12) Patent Application Publication
(10) Pub. No.: US 2020/0268242 A1
(43) Pub. Date: Aug. 27, 2020

(54) PREPARING AND MAPPING A TUBE-SHAPED STRUCTURE

(71) Applicant: Siemens Healthcare GmbH, Erlangen (DE)

(72) Inventors: Jessica Magaraggia, Erlangen (DE); Maddalena Strumia, Forchheim (DE); Stefan Britzen, Buckenhof (DE)

(21) Appl. No.: 16/801,122

(22) Filed: Feb. 25, 2020

(30) Foreign Application Priority Data
Feb. 26, 2019 (DE) 102019202571.4

(51) Int. Cl. A61B 1/267; A61B 1/00; A61B 6/03; A61B 6/00; A61M 25/10

(52) U.S. Cl. CPC A61B 1/2676; A61B 1/0011; A61M 25/10181; A61B 6/485; A61B 1/00082; A61B 6/032

(57) ABSTRACT

In order to improve the detectability of a structure that is tube-shaped in an imaging method, a method for preparing the structure is specified. A first channel is formed inside an inner wall of an auxiliary device, and a second channel is formed between the inner wall and an outer wall. A first fluid that contains a material for contrast amplification is poured into the structure via the first channel. The first channel is then closed by a swelling element of the auxiliary device being enlarged by pouring a second fluid into the second channel.

This application claims the benefit of German Patent Application No. DE 10 2019 202 571.4, filed Feb. 26, 2019, which is incorporated by reference in its entirety.

### BACKGROUND

The present embodiments relate to a method for preparing a tube-shaped structure before an imaging method, a corresponding auxiliary device, a method for mapping at least one tube-shaped structure, and a corresponding system.

Imaging methods, such as computer tomography methods, for example, may be used in a medical context. For example, imaging methods may be used during an operation (e.g., intraoperatively). Imaging methods may also be used for examining workpieces, materials, or other objects (e.g., non-organic or non-living objects).

When examining tube-shaped structures (e.g., small, tube-shaped and possibly branched structures, such as a bronchial system during an operation or when examining branched, non-organic, tube-shaped structures), it may be necessary or useful to support navigation through the tube-shaped structure using an imaging method. An example of this is an intervention (e.g., a biopsy relating to the lungs), in which the bronchi or the bronchial system are accessed by a bronchoscope.

Depending on the nature and material of the tube-shaped structure, however, it may be difficult or impossible to adequately map this using the known imaging methods. For example, if the tube-shaped structures are peripheral or small bronchi or bronchioles with a small diameter and/or a small wall thickness, the tube-shaped structures may not be discernible or barely discernible in known imaging methods, such as, for example, computer tomography methods. This also applies, for example, to cone beam computer tomography methods and fluorescence-based computer tomography methods.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved way for preparing and mapping a tube-shaped structure, by which improved detectability of small structures in imaging is achieved, is provided.

One or more of the present embodiments are based on the idea of introducing a material for contrast enhancement through a first channel into a tube-shaped structure by an auxiliary device that has already been introduced into the tube-shaped structure, and by a fluid that is poured into a second channel to use a swelling element of the auxiliary device for closing the auxiliary device.

According to a first aspect, a method for preparing at least one tube-shaped structure is provided (e.g., for preparing the structure before an imaging method). Prior to the preparation method, an auxiliary device was introduced into the tube-shaped structure. According to the method, a first channel is formed within an inner wall of the auxiliary device, and a second channel is formed between the inner wall and an outer wall of the auxiliary device. A first fluid, which contains a material for contrast enhancement, is poured into the tube-shaped structure through the first channel. The first channel is then closed by a swelling element of the auxiliary device being enlarged by pouring a second fluid into the second channel.

The term “tube-shaped” may be elongated and hollow in the medical (e.g., anatomical) context, as well as generally in the context of the present embodiments. In the context of anatomy, “tubes” refer to parts of accordingly elongated hollow organs. “Tube-shaped” may be that the tube-shaped structure is suitable for conveying, forwarding, or storing solid, liquid, or gaseous substances.

That the first channel is formed within the inner wall may be that the first channel is formed by an area of the auxiliary device that is enclosed by the inner wall (e.g., in a pipe-shaped, tube-shaped or channel-shaped manner).

For example, the method prepares an inner area, an interior, or one or more inner walls of the tube-shaped structure.

The tube-shaped structure may include a single tube-shaped structure or two or more connected and/or branched tube-shaped structures (e.g., a network of tube-shaped structures). For example, the tube-shaped structure may be a bronchial system or part of a bronchial system (e.g., one or more bronchi and/or bronchioles).

The structure may include one or more networked or connected cavities. The cavity or cavities may be at least partially filled with a gas or a liquid. Walls or boundaries of the structure may be rigid or flexible.

The auxiliary device (e.g., the first channel) has a first opening at a first end of the auxiliary device through which the first fluid may be poured into the first channel. For example, the auxiliary device (e.g., the first channel) also has a second opening at a second end of the auxiliary device opposite the first end of the auxiliary device, by which the first fluid poured into the first channel may pass from the auxiliary device into a further area in the tube-shaped structure. For example, prior to the method for preparation with the second opening, the auxiliary device was first introduced into the tube-shaped structure.

The enlargement of the swelling element may, for example, be a radial enlargement of the swelling element in relation to a longitudinal axis of the auxiliary device (e.g., of the first channel and the second channel).

For example, the swelling element may include a flexible material or an expandable material (e.g., an expandable membrane) that expands as a result of the pouring of the second fluid and leads to the enlargement of the swelling element. For example, the swelling element may be a balloon (e.g., a catheter balloon such as a Fogarty balloon). By pouring the second fluid into the second channel, the swelling element is therefore inflated, filled, or pumped up, for example.

The first channel is closed, for example, by locally narrowing the first channel through enlarging the swelling element to such an extent that opposite parts of the swelling element and/or the inner wall of the auxiliary device touch and are pressed together.

The first channel is closed, for example, such that the first fluid may no longer flow back through the first channel past the swelling element.

The second fluid may include, for example, a gas (e.g., air or nitrogen) or a liquid (e.g., water, purified water, ultrapure water, or a salt solution).

The second channel, for example, completely surrounds the first channel at least in a partial area along the longitudinal axis of the auxiliary device.

The method for preparation may, for example, be used in a medical context (e.g., intraoperatively). However, the method may also be used for non-medical (e.g., non-surgical), non-therapeutic, and non-diagnostic purposes (e.g., when examining materials or other objects such as non-organic objects).

The fact that the auxiliary structure has already been introduced into the tube-shaped structure before the method for preparation according to the present embodiments provides that at the beginning of the method for preparation, the auxiliary device is already located within or partially within the tube-shaped structure and, for example, cannot and need not be moved during the method for preparation. The first channel may be used before or after the preparation of the tube-shaped structure for the introduction of a tool (e.g., an endoscope or bronchoscope) into the tube-shaped structure.

Using the method for preparing the tube-shaped structure (e.g., by introducing the first fluid with the material for contrast enhancement and closing the first channel), the contrast enhancement is applied locally to a limited extent in the tube-shaped structure. There, the contrast enhancement may be used for advantageous contrast enhancement when using a subsequent imaging method.

By closing the first channel, only a limited quantity of the first fluid is to be used, as the entire network of tube-shaped structures (e.g., a large part of the bronchial system) need not nor should not be filled with the first fluid. For example, this may be advantageous in the case of intraoperative interventions, as the contrast-enhancing material may contain harmful or irritating substances. When examining non-organic objects, limited contact of the structure with the contrast-enhancing material may also be advantageous (e.g., in order to minimize the interaction of the contrast-enhancing material with the structure to be examined as much as possible).

By applying the contrast-enhancing material, better visibility or detectability of the structure may be achieved in a subsequent imaging method, or any detectability of the structure in the imaging at all may be achieved. Depending on the type of imaging method, lower demands may also be made on the resolution of the imaging method, as the lack of resolution may be compensated, for example, at least partially by higher contrasts.

In comparison with preoperative fluorescence-assisted methods in which, for example, fluorescent substances are used before an operation to perform the imaging before the operation, a preparation according to the improved concept has the advantage that an imaging method based thereon may take place in real time (e.g., during the operation or the examination of the non-organic object). This implies greater accuracy of the imaging method, as, for example, during processing, examination, treatment, or operation of the tube-shaped structure, the tube-shaped structure may be altered, damaged, or injured. This may be taken into account in real time by the imaging in accordance with the present embodiments.

According to at least one embodiment, the method is a method for preparing a non-organic or non-living, tube-shaped structure (e.g., before an imaging method that is not carried out for medical purposes, such as surgical, therapeutic, or diagnostic purposes; not on a human or an animal). In such embodiments, the method act of introducing the auxiliary device into the tube-shaped structure may be part of the method for preparing the tube-shaped structure.

According to at least one embodiment, a liquid or gaseous contrast agent, a fluorogenic material, a fluorophore material, a fluorescent material, or a salt solution is used as material for contrast enhancement.

The liquid or gaseous contrast agent is, for example, a fluorescent contrast agent.

Here and hereinafter, the terms “fluorogenic”, “fluorophore”, and “fluorescent” are used interchangeably and identically, and in each case, provide that the relevant material or substance has the property of fluorescence. For example, this does not provide that the corresponding material emits fluorescent light during filling.

The salt solution may be a sodium chloride solution or another salt solution (e.g., a salt solution that is harmless to health).

The contrast-enhancing material may also be identical to the first fluid (e.g., in the case of a salt solution).

An advantage of embodiments that include use of the salt solution may be, for example, that salt solutions may be harmless to health (e.g., during operations or interventions in the bronchial system).

Embodiments in which a gaseous contrast agent (e.g., a gaseous fluorophore) is used have the advantage, for example, that the gas may be particularly easily removed from or pumped out of the tube-shaped structure (e.g., the lungs or the bronchial system).

Embodiments in which liquid contrast agents are used result in higher achievable contrasts in the subsequent imaging method.

According to at least one embodiment, the tube-shaped structure is closed by the swelling element being enlarged by the pouring of the second fluid into the second channel such that the outer wall of the auxiliary device or an outer wall of the swelling element fits closely against an inner wall of the tube-shaped structure.

The closing of the tube-shaped structure may be achieved, for example, by closing the first channel and enlarging the swelling element such that the outer wall fits closely against the inner wall of the structure.

The closing of the tube-shaped structure provides, for example, that a part of the tube-shaped structure is sealed or closed in at least one direction.

For example, by using the flexible or expandable material of the swelling element (e.g., the balloon or the membrane), a reliable and complete sealing of the tube-shaped structure may be achieved.

According to at least one embodiment, the closing of the tube-shaped structure prevents the first fluid from reaching another part of the tube-shaped structure from a part of the tube-shaped structure that is closed by closing the tube-shaped structure.

It is advantageous in such embodiments (e.g., in lung operations or interventions on the bronchial system) that the first fluid cannot flow in bronchi or bronchioles or parts of the bronchial system where the first fluid is not required. This may be advantageous, for example, in the case of contrast-enhancing materials that are harmful to health.

Due to the fact that the outer wall of the auxiliary structure or of the swelling element fits closely against the inner wall of the tube-shaped structure and the first channel is likewise closed, a pressure is exerted on the inner wall of the tube-shaped structure by closing the first channel via the swelling element so that the closing of the tube-shaped structure is assisted thereby.

By preventing the first fluid from entering the other part of the tube-shaped structure, it is possible to reduce the amount of contrast-enhancing material used.

According to at least one embodiment, at least one bronchus and/or at least one bronchiole is prepared by the method for preparation.

In such embodiments, the method for preparation may, for example, support the navigation necessary for the operation, for example, of a bronchoscope.

For example, the auxiliary device may have been introduced into the tube-shaped structure as a precaution before the operation. Thus, the method for preparation and, if necessary, an imaging method may be carried out during the operation, as required.

According to a further aspect, a method for mapping at least one tube-shaped structure is also provided. An auxiliary device was introduced into the tube-shaped structure before the method for mapping. The method for mapping includes the preparation of the tube-shaped structure (e.g., an inner area of the tube-shaped structure) by a method for preparing a tube-shaped structure according to the present embodiments. The method for mapping at least one tube-shaped structure also includes, after preparation, performing an imaging method for mapping the tube-shaped structure using the contrast enhancement by the material of the first fluid.

In this context, the use of contrast enhancement provides that the material for contrast enhancement is matched to the imaging method such that a relevant contrast may be increased in the imaging method. Mapping of the tube-shaped structure is achieved by the imaging method.

Any imaging method in which a contrast agent or a contrast-enhancing material may be used (e.g., a fluorescence-assisted imaging method) may be used for a method for mapping according to the present embodiments.

According to at least one embodiment of the method for mapping, after performing the imaging method, the first fluid is at least partially removed from the tube-shaped structure via the first channel.

For example, the first fluid may be aspirated and/or drained.

For example, this may be advantageous if the use of the first fluid or of the contrast-enhancing material entails a health risk for a patient.

According to at least one embodiment, the imaging method includes a computer tomography method, a cone beam computer tomography (CBCT) method, a fluorescence-based computer tomography method, a fluorescence-assisted computer tomography method, a digital subtraction angiography (DSA) method, or a magnetic resonance tomography (MRT) method.

Such imaging methods may be supported and improved particularly well by the method for preparation.

The term “angiography method” is used in connection with vascular examinations. Corresponding methods may, however, also be used in an analogous manner for the examination of a bronchial system (e.g., of bronchi or bronchioles).

According to a further aspect, an auxiliary device for preparing at least one tube-shaped structure before an imaging method is provided. The auxiliary device has an inner wall, an outer wall, and a first channel formed within the inner wall. A second channel is formed between the inner and the outer wall. A first fluid may be poured into the first channel (e.g., when the auxiliary device has been introduced into the tube-shaped structure). The auxiliary device has a swelling element that may be enlarged by pouring a second fluid into the second channel. The first channel may be closed by the enlargement of the swelling element.

The auxiliary device may, for example, be a catheter, a catheter device, or a tube-shaped, tubular, or pipe-shaped device.

By enlarging the swelling element, opposite parts of an inner wall of the swelling element or parts of the inner wall of the auxiliary device approach each other so that a corresponding inner diameter is reduced, and if the swelling element is sufficiently enlarged, the first channel is finally closed. The swelling element may, for example, be arranged or shaped in an annular manner.

When enlarging the swelling element, when the auxiliary device has been introduced into the tube-shaped structure, the auxiliary device is moved towards an inner wall of the tube-shaped structure such that a distance between the swelling element and the inner wall of the structure is reduced. Depending on the diameter of the structure and maximum enlargement of the swelling element, the distance disappears when the outer wall of the swelling element fits closely against the inner wall of the tube-shaped structure.

In order to pour the first fluid into the tube-shaped structure, the inner channel has a first opening at a first end of the auxiliary device and a second opening at a second end opposite the first end. Using the first opening, the first fluid may be poured into the auxiliary device; the first fluid may then pass further into the tube-shaped structure through the second opening.

The outer channel is at least partially opened at the first end of the auxiliary device, for example, such that the second fluid may be poured in. The second channel is closed at the second end of the auxiliary device, for example, so that pressure may build up on the swelling element as a result of the second fluid in order to enlarge the swelling element.

According to at least one embodiment of the auxiliary device, the swelling element has a Fogarty balloon.

According to at least one embodiment, the auxiliary device has an expandable membrane that is fastened to the inner wall and to the outer wall of the auxiliary device such that a balloon (e.g., an annular balloon) is formed as a swelling element.

The expandable membrane is configured, for example, as an annular strip. An edge of the strip is fastened, for example, to an outer wall of the auxiliary device. A second edge of the strip is fastened, for example, to the inner wall of the auxiliary device (e.g., at a position that is closer to the second opening of the auxiliary device than the first edge of the strip).

The expandable membrane thus forms part of the outer wall of the auxiliary device or continues the outer wall. The membrane thus forms or continues a part of the second channel.

According to at least one embodiment, the auxiliary device has a pump unit that is configured to pump the first fluid for pouring into the first channel and/or to pump the second fluid for pouring into the second channel.

The pump unit may include, for example, one or more pumps (e.g., manually operable or automatically operable pumps). The pump unit may also have a connection device for connecting the one or more pumps to the first channel and/or the second channel.

According to at least one embodiment, the auxiliary device has a further pump unit that is configured to at least partially remove the first fluid from the tube-shaped structure via the first channel.

In various embodiments, the pump unit may also be configured to at least partially remove the first fluid from the tube-shaped structure via the first channel. In such embodiments, the additional pump unit is not essential.

According to a further aspect, a system for mapping at least one tube-shaped structure is provided. The system has a device for performing an imaging method and an auxiliary device according to the present embodiments.

The device for performing an imaging method may have, for example, a computer tomography device, a magnetic resonance tomography device, a microscope, an operating microscope, a device for digital subtraction angiography, or the like.

Further embodiments of the auxiliary device and the system are obtained from the various embodiments of the method for preparing at least one tube-shaped structure according to the present embodiments, and vice versa, and from the various embodiments of the method for mapping at least one tube-shaped structure, and vice versa.

### BRIEF DESCRIPTION OF THE DRAWINGS

In the figures, same or functionally same elements are provided with the same reference symbols.

The description of same or functionally same elements is, if appropriate, not necessarily repeated in different figures. The diagrams show:

FIG. 1 is a diagrammatic view of an exemplary embodiment of an auxiliary device;

FIG. 2 is a diagrammatic view of a further exemplary embodiment of an auxiliary device for use in a method for preparing at least one tube-shaped structure; and

FIG. 3 is a diagrammatic view of an exemplary embodiment of a system for mapping at least one tube-shaped structure.

### DETAILED DESCRIPTION

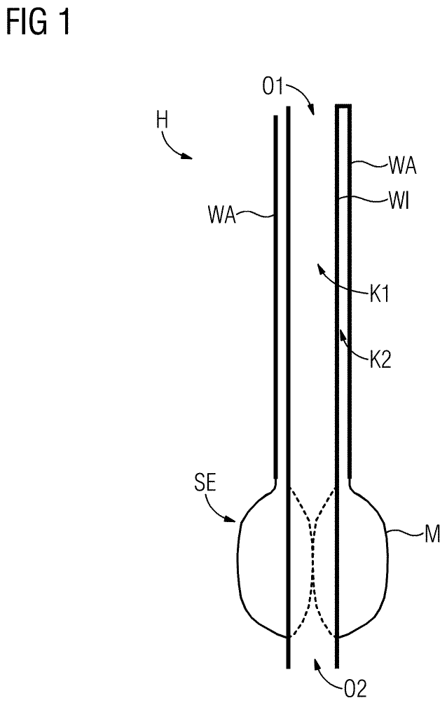

FIG. 1 shows a diagrammatic view (e.g., a longitudinal section) of an exemplary embodiment of an auxiliary device H for preparing at least one tube-shaped structure before an imaging method according to one or more of the present embodiments.

The auxiliary device H is, for example, tube-shaped, pipe-shaped, or channel-shaped and, for example, double-walled. For example, the auxiliary device H has an outer wall WA and an inner wall WI. The outer wall and the inner wall may each contain a pipe-shaped material (e.g., plastic material) that is, for example, a flexible or deformable material.

The inner wall WI forms a first channel K1 of the auxiliary device H inside the inner wall WI. An area between the inner wall WI and the outer wall WA is, for example, empty. As a result, a second channel K2 is formed between the inner wall and the outer wall WI, WA. A first opening O1 of the inner channel WI and the auxiliary device H are located at a first end of the auxiliary device H. A second opening O2 of the inner channel WI and the auxiliary device H is located at an opposite end of the auxiliary device H.

The auxiliary device H also has a swelling element SE. The swelling element SE includes, for example, an expandable membrane M that is attached and fastened to the inner wall WI and the outer wall WA such that a balloon (e.g., an annular balloon) is formed.

The membrane M is, for example, configured as an annular strip. A first edge (e.g., a circular edge) of the membrane M faces the first opening O1 and, for example, is fastened (e.g., in a fluid-impermeable manner) to the outer wall WA. On a second edge opposite the first edge (e.g., an annular edge), the membrane M is fastened (e.g., in a fluid-impermeable manner) to the inner wall WI. The attachment of the membrane M to the inner wall WI is, for example, at a location closer to the second opening O2 than an attachment point of the membrane M to the first wall WI. For example, the second edge of the membrane M faces the second opening O2.

In an area between the attachment points of the membrane M to the inner and the outer wall WI, WA, the outer wall WA and the second channel K2 are continued through the membrane M. In other words, the membrane M forms part of the outer wall WA in this area. For example, the auxiliary device H does not have an outer wall in an area between the second opening O2 and the attachment point of the membrane M to the inner wall WI, apart from the membrane M, and is therefore, for example, not double-walled, except for the membrane M.

The swelling body SE thus forms a balloon that may be enlarged (e.g., inflated) by pouring a second fluid into the second channel K2. As a result, the annular balloon is enlarged outwards and inwards. For example, with sufficient enlargement, the first channel K1 may be completely closed, as indicated by dashed lines.

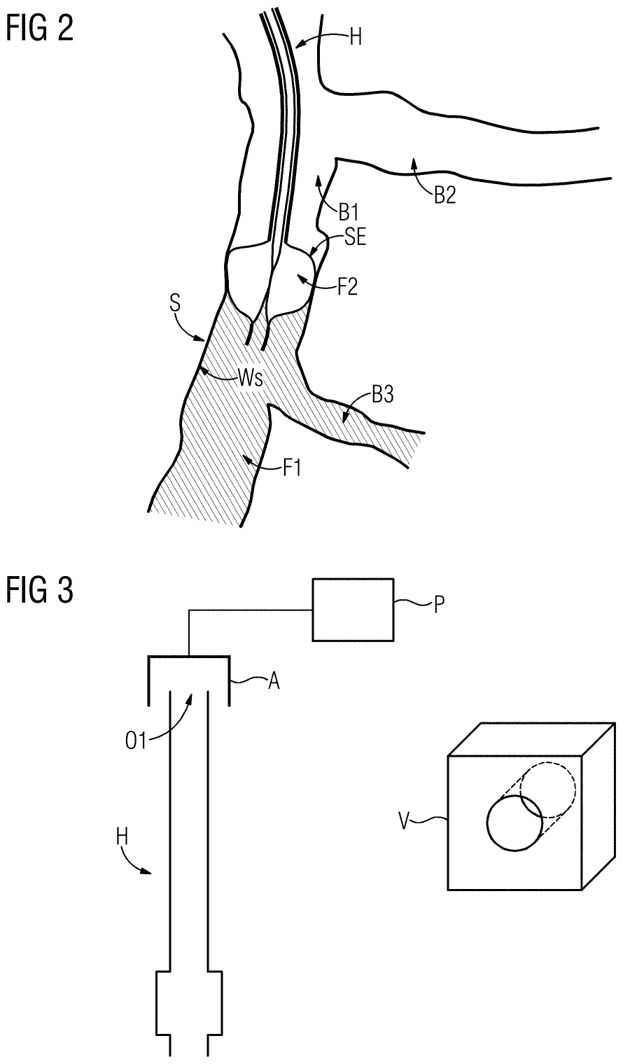

FIG. 2 shows an exemplary embodiment of an auxiliary device H (e.g., the auxiliary device H from FIG. 1) in use (e.g., in the preparation of a tube-shaped structure S) according to an exemplary embodiment of a method.

The tube-shaped structure S may be, for example, part of a bronchial system of a human being. The bronchial system may have, for example, a first bronchus B1, a second bronchus B2 branching therefrom, and a bronchus B3 branching off from the first bronchus B1 at another location.

The structure of the bronchus or the bronchial system or the tube-shaped structure shown is not meant to be restrictive and is purely for illustrative purposes.

Before the start of the method for preparation, the auxiliary device H was already introduced into the tube-shaped structure S. This may have been done, for example, in order to be able to perform a method for preparing or mapping the tube-shaped structure S according to one or more of the present embodiments during intraoperative imaging and/or to be able to introduce a tool (e.g., a bronchoscope) through the auxiliary device H into the bronchial system.

The swelling body SE of the auxiliary device H is positioned by introduction into the tube-shaped structure S, for example, such that the swelling body SE is located at a level between a branch of the bronchus B3 and a branch of the bronchus B2 from the bronchus B1.

After the auxiliary device H has been introduced into the tube-shaped structure S, a first fluid F1 (e.g., a liquid contrast agent or a salt solution) is poured into the tube-shaped structure S via the first channel of the auxiliary device H when the swelling element is not or not substantially enlarged (e.g., when the balloon was not inflated), which is illustrated by hatching in Figure. 2. As a result, both areas of the bronchus B1 below the swelling element SE and the bronchus B3 or parts of the bronchus B3 are filled with the first fluid.

After the introduction of the first fluid F1, a second fluid F2 is poured into the balloon via the second channel of the auxiliary device H. As a result, the balloon is enlarged to such an extent that opposite parts of the inner wall WI are pressed together so that the first channel is closed; further, the membrane M is pressed against an inner wall of the structure WS so that the membrane M fits closely against the inner wall WS.

As a result of the enlargement of the swelling element SE, the part of the bronchus B1 and of the bronchus B3 filled by the first fluid F1 is consequently separated from other parts of the bronchus B1 and, for example, from the bronchus B2. The corresponding part in the tube-shaped structure S is therefore closed.

In the state shown in FIG. 2, for example, an imaging method (e.g., computer tomography, cone beam computer tomography, or fluorescence-assisted computed tomography) may be carried out in order to obtain an image of the tube-shaped structure S in the areas filled by the first fluid F1. The first fluid F1, the contrast agent, or the salt solution leads to enhanced contrasts in a result of the imaging method.

For this purpose, contrast enhancement by a liquid contrast agent (e.g., a standard contrast agent used in imaging methods) may be particularly high (e.g., higher than in the case of a salt solution). In the case of a salt solution, an advantage may be that the salt solution is harmless to the health of the patient. The corresponding advantages and disadvantages may be balanced with respect to one another according to the present embodiments.

FIG. 3 shows a diagrammatic view of one embodiment of a system.

The system includes an auxiliary device H and a device V for carrying out an imaging method. The device V may be, for example, a computer tomography device or a magnetic resonance tomography device.

The system optionally includes a pump unit P (e.g., a pump) and an optional connection device A. The pump unit P may be fastened to the first opening O1 of the auxiliary device H by the connection device A. For example, the connection device A may be configured such that the first fluid may be pumped or poured into the first channel of the auxiliary device H and thus into the tube-shaped structure by the pump unit P. Alternatively or additionally, after carrying out the imaging method, the first fluid may be removed at least partially by the pump unit P via the first channel from the tube-shaped structure (e.g., drained or aspirated).

After performing the imaging method, the swelling element (e.g., the balloon) may be vented or deflated to cancel the closure of the first channel and the tube-shaped structure S. Thereafter, for example, the first fluid may be removed completely or partially via the first channel (e.g., drained).

According to one or more of the present embodiments, using, for example, an auxiliary device or a method according to one or more of the present embodiments, the visibility (e.g., of small tube-shaped structures; of peripheral bronchi or bronchioles) may be improved or even enabled in an imaging method. This may considerably simplify contrast-enhanced navigation (e.g., during an operation).

The improved concept enables real-time recording by the imaging method during the operation. Changes that occur shortly before or during the operation may thus be taken into account. In addition, the influence of possibly limited resolution in imaging methods is of less importance.

Various embodiments of the auxiliary device and the method use the first channel as a working channel by which, for example, tools such as, for example, a bronchoscope, to which an inflatable or fillable balloon is fastened, may be introduced into the structure. When the balloon is filled, an outer side of the balloon is placed against the tissue or the interior of the tube-shaped structure, while an inner side of the balloon closes the working channel.

For example, according to the present embodiments, structures such as airways of the lungs, which in conventional approaches, are too small to be visible in an imaging process, may still be mapped. The airways are only made visible in accordance with the present embodiments. The airways may, for example, also be segmented as part of the imaging method and used for further processing.

Another advantage of one or more of the present embodiments is that during an operation a doctor may more quickly ascertain along which airways the doctor is to navigate the bronchoscope or other tool as a result of the improved detectability.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for preparing a structure that is tube-shaped before an imaging method, wherein an auxiliary device has previously been introduced into the structure, the method comprising:
forming a first channel within an inner wall of the auxiliary device and forming a second channel between the inner and an outer wall of the auxiliary device;
pouring a first fluid, which contains a material for contrast enhancement, into the structure through the first channel; and
closing the first channel, the closing of the first channel comprising enlarging a swelling element of the auxiliary device, the enlarging of the swelling element comprising pouring a second fluid into the second channel.

2. The method of claim 1, wherein a liquid or gaseous contrast agent, a fluorogenic material, or a salt solution is used as the material for contrast enhancement.

3. The method of claim 1, wherein the structure is closed through enlarging the swelling element by pouring the second fluid into the second channel such that the outer wall of the auxiliary device fits against an inner wall of the structure.

4. The method of claim 3, wherein closing the structure prevents the first fluid from flowing from one closed part of the structure into another part of the structure.

5. The method of claim 1, wherein at least one bronchus, at least one bronchiole, or at least one bronchus and at least one bronchiole are prepared by the method.

6. A method for mapping a tube-shaped structure, wherein an auxiliary device has previously been introduced into the tube-shaped structure, the method comprising:
preparing the tube-shaped structure before an imaging method, the preparing of the tube-shaped structure comprising:
forming a first channel within an inner wall of the auxiliary device and forming a second channel between the inner and an outer wall of the auxiliary device;
pouring a first fluid, which contains a material for contrast enhancement, into the tube-shaped structure through the first channel; and
closing the first channel, the closing of the first channel comprising enlarging a swelling element of the auxiliary device, the enlarging of the swelling element comprising pouring a second fluid into the second channel; and
performing the imaging method for mapping the tube-shaped structure using the contrast enhancement by the material of the first fluid.

7. The method of claim 6, further comprising at least partially removing the first fluid from the tube-shaped structure via the first channel after carrying out the imaging method.

8. The method of claim 6, wherein the imaging method comprises a computer tomography method, a cone beam computer tomography method, a fluorescence-based computer tomography method, a digital subtraction angiography method, or a magnetic resonance tomography method.

9. An auxiliary device for preparing at least one tube-shaped structure before an imaging method, wherein the auxiliary device has an inner wall, an outer wall, and a first channel formed within the inner wall, the auxiliary device comprising:
a second channel formed between the inner wall and the outer wall, wherein a first fluid is pourable into the first channel when the auxiliary device has been introduced into the at least one tube-shaped structure; and
a swelling element that is enlargeable when a second fluid is poured into the second channel,
wherein the first channel is closeable by enlarging the swelling element.

10. The auxiliary device of claim 9, wherein the swelling element includes a Fogarty balloon.

11. The auxiliary device of claim 9, further comprising an expandable membrane that is fastened to the inner wall and the outer wall such that a balloon is formed as the swelling element.

12. The auxiliary device of claim 9, further comprising a pump configured to:
pump the first fluid for pouring into the first channel;
pump the second fluid for pouring into the second channel; or
pump the first fluid for pouring into the first channel and pump the second fluid for pouring into the second channel.

13. The auxiliary device of claim 9, further comprising a pump configured to at least partially remove the first fluid from the tube-shaped structure via the first channel.

14. A system for mapping at least one tube-shaped structure, the system comprising:
a device for carrying out an imaging method; and
an auxiliary device for preparing the at least one tube-shaped structure before the imaging method, wherein the auxiliary device has an inner wall, an outer wall, and a first channel formed within the inner wall, the auxiliary device comprising:
a second channel formed between the inner wall and the outer wall, wherein a first fluid is pourable into the first channel when the auxiliary device has been introduced into the at least one tube-shaped structure; and
a swelling element that is enlargeable when a second fluid is poured into the second channel,
wherein the first channel is closeable by enlarging the swelling element.

15. The system of claim 14, wherein the swelling element includes a Fogarty balloon.

16. The system of claim 14, wherein the auxiliary device further comprises an expandable membrane that is fastened to the inner wall and the outer wall such that a balloon is formed as the swelling element.

17. The system of claim 14, wherein the auxiliary device further comprises a pump configured to:
pump the first fluid for pouring into the first channel;
pump the second fluid for pouring into the second channel; or
pump the first fluid for pouring into the first channel and pump the second fluid for pouring into the second channel.

18. The system of claim 14, wherein the auxiliary device further comprises a pump configured to at least partially remove the first fluid from the tube-shaped structure via the first channel.
